(19) United States
(12) Patent Application Publication
(10) Pub. No.: US 2010/0166722 A1
(43) Pub. Date: Jul. 1, 2010

(54) T CELL THERAPIES

(75) Inventors: Alan David Bennett, Abingdon (GB); Bent Karsten Jakobsen, Abingdon (GB)

Correspondence Address: BANNER & WITCOFF, LTD., 1100 13th STREET, N.W., SUITE 1200, WASHINGTON, DC, 20005-4051, US

(73) Assignee: IMMUNOCORE LTD., Abingdon, OX (GB)

(21) Appl. No.: 12/443,078

(22) PCT Filed: Apr. 3, 2008
(86) PCT No.: PCT/GB07/03676

§ 371 Date: Apr. 15, 2009

(30) Foreign Application Priority Data
Sep. 29, 2006 (GB) 0619251.2
Feb. 22, 2007 (GB) 0703406.9

(51) Int. Cl. A61K 35/26; A61P 35/00; A61P 31/00

(52) U.S. Cl. 424/93.71

(57) ABSTRACT

This invention provides a method of treating cancer or infection by administering T cells transfected with T cell receptors (TCRs) which in their soluble form have a half life for their interaction with their cognate peptide-MHC complex chosen to enhance the avidity of the T cells for target cells presenting that peptide MHC complex while maintaining the activation specificity of the T cells by that peptide-MHC complex.

This invention relates to a method of treating cancer or infection by administering T cells transfected with T cell receptors (TCRs) which in their soluble form have a half life for their interaction with their cognate peptide-MHC complex chosen to enhance the avidity of the T cells for target cells presenting that peptide MHC complex while maintaining the activation specificity of the T cells by that peptide-MHC complex.

### BACKGROUND TO THE INVENTION

Immunotherapy involves enhancing the immune response of a patient to cancerous or infected cells. Active immunotherapy is carried out by stimulation of the endogenous immune system of tumour bearing patients. Passive, or adoptive, immunotherapy involves the transfer of immune competent cells into the patient. (Paul (2002) Curr Gene Therapy 2: 91-100) There are three broad approaches to adoptive immunotherapy which have been applied in the clinic for the treatment of metastatic diseases; lymphokine-activated killer (LAK) cells, auto-lymphocyte therapy (ALT) and tumour-infiltrating lymphocytes (TIL). (Paul (2002) Curr Gene Therapy 2: 91-100).

One variation of T cell adoptive therapy is the use of gene therapy techniques to introduce TCRs specific for known cancer-specific MHC-peptide complexes into the T cells of cancer patients. For example, WO 01/55366 discloses retrovirus-based methods for transfecting, preferably, T cells with heterologous TCRs. This document states that these transfected cells could be used for either the cell surface display of TCR variants as a means of identifying high affinity TCRs or for immunotherapy. Methods for the molecular cloning of cDNA of a human p53-specific, HLA restricted murine TCR and the transfer of this cDNA to human T cells are described in published US patent application no. 20020064521. This document states that the expression of this murine TCR results in the recognition of endogenously processed human p53 expressed in tumour cells pulsed with the p53-derived peptide 149-157 presented by HLA A*0201 and claims the use of the murine TCR in anti-cancer adoptive immunotherapy. However, the concentration of peptide pulsing required achieving half maximal T cell stimulation of the transfected T cells was approximately 250 times that required by T cells expressing solely the murine TCR. As the authors noted “The difference in level of peptide sensitivity is what might be expected of a transfectant line that contained multiple different TCR heterodimers as a result of independent association of all four expressed hu and mu TCR chains.”

There are also a number of papers relating to T cell adoptive therapy. In one study (Rosenberg (1988) N Engl J Med 319 (25): 1676-80) lymphocytes from melanomas were expanded in vitro and these tumor-infiltrating lymphocytes, in combination with IL-2 were used to treat 20 patients with metastatic melanoma by means of adoptive transfer. The authors note that objective regression of the cancer was observed in 9 of 15 patients (60 percent) who had not previously been treated with interleukin-2 and in 2 of 5 patients (40 percent) in whom previous therapy with interleukin-2 had failed. Regression of cancer occurred in the lungs, liver, bone, skin, and subcutaneous sites and lasted from 2 to more than 13 months. A further study describes the administration of an expanded population of Melan-A specific cytotoxic T cells to eight patients with refractory malignant melanoma. These T cells were administered by i.v. infusion at fortnightly intervals, accompanied by s.c. administration of IL-2. The T cell infusions were well tolerated with clinical responses noted as one partial, one mixed with shrinkage of one metastatic deposit and one no change (12 months) among the eight patients. (Meidenbauer (2003) J Immunol 170: 2161-2169) As noted in this study, recent advances regarding the in vitro stimulation T cells for the generation of cell populations suitable for T cell adoptive therapy have made this approach more practical. See, for example (Oelke (2000) Clin Cancer Res 6: 1997-2005) and (Szmania (2001) Blood 98: 505-12).

It has therefore been recognised in the art that it would be desirable to improve the immune response of T-cells administered in adoptive therapy. The expectation has been that T-cells transfected with TCRs having high affinities for their cognate p-MHCs would produce the desired improvement in immune response.

It has recently become possible to create TCRs having high and specific in vitro affinities for their respective pMHCs. Phage display provides one means by which libraries of TCR variants can be generated. Methods suitable for the phage display and subsequent screening of libraries of TCR variants each containing a non-native disulfide interchain bond are detailed in (Li et al., (2005) Nature Biotech 23 (3): 349-354) and WO 2004/04404.

Holler et. al. 2001 J. Exp. Med, 194, (8):1043-1052 states that various models have predicted that activation (of T-cells) is limited to a narrow window of TCR characteristics such as affinities, dissociation rates, half life of interaction and the like, for the TCR-pMHC interaction, and the affinity of the antigenic peptide for its cognate MHC, and that above or below this window the T-cells will fail to undergo activation. However, the paper comes to no definitive conclusion regarding which is the controlling parameter defining that “window” or the values that define said window. The paper reports that in some experiments using CD8-T-cell hybridomas transfected with a high affinity (K_{D}=10 nM) mutant TCR activation could be detected at lower peptide concentrations than with T-cell hybridomas expressing the wild type TCR. The paper states, based on those results: “ . . . It also may indicate that the in-vitro engineering of TCRs for higher affinity could prove useful for increasing the activity of T cells against particular pMHC targets, for example in adoptive T cell therapies.”

Holler et. al. (2003) Nat. Immunol. 4 (1): 55-62 showed that T-cells transfected with a high affinity TCR were non-specifically activated, and therefore potentially dangerously autoreactive. The paper concludes “It is possible that in vitro engineering of TCRs with precise affinities against self or foreign pMHC ligands could be performed to optimise this balance between favourable T cell activity and dangerous autoreactivity.

Despite the recognition in the art of the desirability of using high affinity TCRs for transfection of TCRs to increase T cell-mediated immune responses, and the assumption in the art that there exists a balance between the level of affinity of a TCR and the loss of specificity of activation of the transfected T-cell by the TCR's cognate pMHC, no studies have been reported which seek to map the range of affinities of TCRs which avoid non-specific activation of the transfected T-cells. Furthermore, although Holler et. al. 2001 J. Exp. Med, 194, (8):1043-1052 refers to theories which rely on other characteristics of the T cell activation process, the art does not establish which characteristic other than affinity may be determinative of increased pMHC-specific T cell mediated immune response.

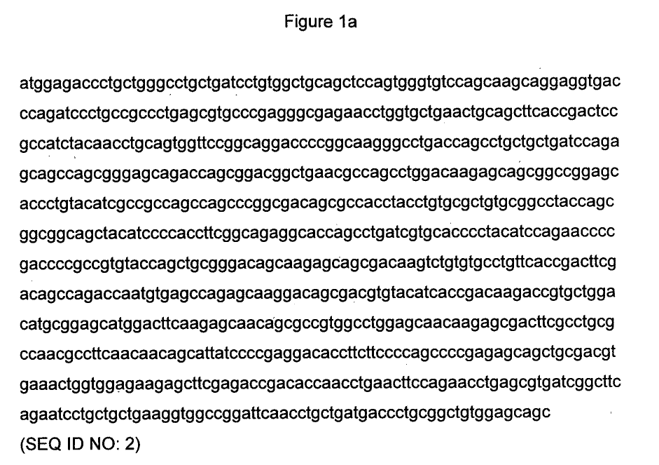

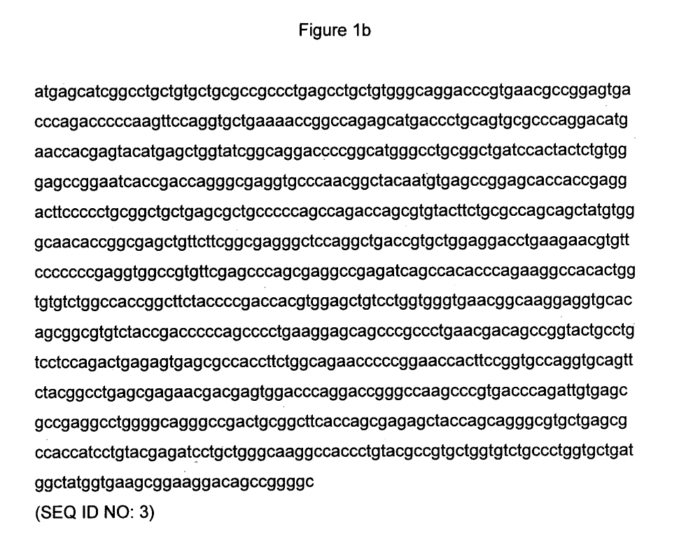

One recent study (Morgan et. al: (2006) Science 314 (5796): 126-129) details the use of autologous lymphocytes transduced ex-vivo with genes encoding a wild-type MART-1 specific TCR to treat 15 patients with metastatic melanoma. Two of these patients demonstrated a sustained regression of their metastatic melanoma as assessed by standard RECIST criteria. This study also states “Engineering PBL to express high affinity TCR recognising the NY-ESO-1 or the p53 antigen, as shown in FIG. 1A and table S1, enables the in-vitro recognition of tumour-associated antigens expressed on a variety of common cancers and the use of these genetically engineered cells for the treatment of patients with common epithelial cancers deserves evaluation”. However, this study does not provide any data regarding their affinity or APC-recognition efficacy in comparison to PBL transfected with the corresponding wild-type TCRs and the high level of peptide pulsing used in this in-vitro experiment (1 μM) would be expected to result in a level of peptide-MHC presentation of the surface of the APCs used which was well above that found in under physiological conditions

### BRIEF DESCRIPTION OF THE INVENTION

This invention is based on the results of experiments which seek to establish the characteristic of the T cell activation process determinative of increased pMHC-specific T cell mediated immune response. The data has shown that pMHC-specific T-cell mediated, immune responses can be enhanced if the T-cells are transfected with TCRs which, in soluble form, have a half life for their interaction with their cognate peptide-MHC ligands in a particular range. This has enabled us to place numerical limits on the effective range of those half lives, thereby identifying TCRs which have half lives slower than a first rate limit, and preferably faster than a second rate limit for use in adoptive T cell therapy. T cells transfected with TCRs not meeting those criteria are unlikely to produce significant T cell mediated immune response or are likely to produce non-specific T cell mediated immune responses.

### DETAILED DESCRIPTION OF THE INVENTION

The present invention provides, in its broadest aspect, a method of treatment of a disease selected from cancer and infection comprising the administration to a subject suffering such disease a plurality of TCR-transfected T cells which are specifically activated by cells presenting a given pMHC characteristic of such disease, at least some of the TCRs presented by each of said T cells having, in soluble form, a half-life for the interaction with the said pMHC which is either:

slower than that of the known corresponding wild type soluble TCR, or
in the case where no corresponding wild-type TCR is known:
(a) 1 second or slower in the case of a class I-restricted TCR transfected into a CD8⁺ T cell, or
(b) 9.6 seconds or slower in the case of a class I-restricted TCR transfected into a CD4⁺ T cell, or in the case of class II-restricted TCR transfected into a CD8⁺ T cell, or
(c) 0.9 seconds or slower in the case of a class II-restricted TCR transfected into a CD4⁺ T cell.

A second aspect of the invention provides the use of a plurality of TCR-transfected T cells which are specifically activated by cells presenting a given pMHC characteristic of cancer or infection, in the preparation of a composition for the treatment of such disease, at least some of the TCRs presented by each of said T cells having, in soluble form, a half-life for the interaction with the said pMHC which is either slower than that of the known corresponding wild type soluble TCR, or in the case where no corresponding wild-type TCR is known:

(a) 1 second or slower in the case of a class I-restricted TCR transfected into a CD8⁺ T cell, or
(b) 9.6 seconds or slower in the case of a class I-restricted TCR transfected into a CD4⁺ T cell, or in the case of class II-restricted TCR transfected into a CD8⁺ T cell, or
(c) 0.9 seconds or slower in the case of a class II-restricted TCR transfected into a CD4⁺ T cell.

In subsidiary embodiments of the invention, in the case where no corresponding wild-type TCR is known, the TCRs presented by each of said T cells has, in soluble form, a half-life for the interaction with the said pMHC which:

(a) in the case of a class I-restricted TCR transfected into a CD8⁺ T cell is preferably 2 seconds or slower, for example 4 seconds or slower, or 9.6 seconds or slower, or
(c) in the case of a class II-restricted TCR transfected into a CD4⁺ T cell, is preferably 2 seconds or slower, for example 4 seconds or slower or 9.6 seconds or slower.

Preferably, TCR transfected T cells for use in the invention include, but are not limited to, those wherein, in addition to having the slower half life limitations mentioned above, at least some of the TCRs presented by said transfected T cells have, in soluble form, a half-life for the interaction with the said pMHC which is either

(a) 12 minutes or faster in the case of a class I-restricted TCR transfected into a CD8⁺ T cell, or
(b) faster than 425 minutes in the case of a class I-restricted TCR transfected into a CD4⁺ T cell, or in the case of class II-restricted TCR transfected into a CD8⁺ T cell, or
(c) 12 minutes or faster in the case of a class II-restricted TCR transfected into a CD4⁺ T cell.

In further subsidiary aspects of the invention, in addition to having the slower half life limitations mentioned above, at least some of the TCRs presented by said transfected T cells have, in soluble form, a half-life for the interaction with the said pMHC which:

(b) in the case of a class I-restricted TCR transfected into a CD4 T cell, or in the case of class II-restricted TCR transfected into a CD8⁺ T cell is preferably 300 minutes or faster, for example 162 minutes or faster, or 12 minutes or faster.

Another aspect of the invention provides a method of treatment of a disease selected from cancer and infection comprising the administration to a subject suffering such disease a plurality of TCR-transfected CD4⁺ and/or CD8⁺ T cells which are specifically activated by cells presenting a given pMHC characteristic of such disease, at least some of the transfected TCRs presented by each of said T cells having, in soluble form, a half-life for the interaction with the said pMHC within the range from 9.6 seconds to 12 minutes. Preferably, said TCRs have, in soluble form, a half-life for the interaction with the said pMHC within the range selected from on of the following:

from 9.6 seconds to 4 minutes, or
from 9.6 seconds to 74 seconds, or
from 9.6 seconds to 41 seconds, or
from 9.6 seconds to 19 seconds, or
from 19 seconds to 12 minutes
from 19 seconds to 4 minutes, or
from 19 seconds to 74 seconds, or
from 19 seconds to 41 seconds, or
from 41 seconds to 12 minutes, or
from 41 seconds to 4 minutes, or
from 41 seconds to 74 seconds, or
from 74 seconds to 12 minutes, or
from 74 seconds to 4 minutes, or
from 4 minutes to 12 minutes.

A further aspect of the invention provides the use of a plurality of TCR-transfected CD4⁺ and/or CD8⁺ T cells which are specifically activated by cells presenting a given pMHC characteristic of cancer or infection, in the preparation of a composition for the treatment of such disease, at least some of the TCRs presented by each of said T cells having, in soluble form, a half-life for the interaction with the said pMHC within the range from 9.6 seconds to 12 minutes. Preferably, said TCRs have, in soluble form, a half-life for the interaction with the said pMHC within the range selected from on of the following:

from 9.6 seconds to 4 minutes, or
from 9.6 seconds to 74 seconds, or
from 9.6 seconds to 41 seconds, or
from 9.6 seconds to 19 seconds, or
from 19 seconds to 12 minutes
from 19 seconds to 4 minutes, or
from 19 seconds to 74 seconds, or
from 19 seconds to 41 seconds, or
from 41 seconds to 12 minutes, or
from 41 seconds to 4 minutes, or
from 41 seconds to 74 seconds, or
from 74 seconds to 12 minutes, or
from 74 seconds to 4 minutes, or
from 4 minutes to 12 minutes.

The TCR transfected T cells used in the invention are either CD3⁺ CD4⁺ “helper” T cells or most commonly, CD3⁺ CD8⁺ “killer” T cells.

Certain preferred embodiments of the present invention are provided wherein the T cell response of said TCR transfected T cells to APCs expressing the peptide-MHC recognised by the transfected TCRs is “enhanced” compared to that of T cells transfected with the corresponding WT TCR. Said “enhanced” response may take the form of an increased T cell response by T cells of the present invention to APCs presenting a fixed level of the cognate peptide-MHC compared to that seen with T cells transfected with the corresponding wild-type TCR and/or an lowering of the level of the cognate peptide-MHC present of the surface of APCs required in order to elicit a T cell response by the T cells of the invention compared to that seen with T cells transfected with the corresponding wild-type TCR. There are a number of methods suitable for measuring this increased T cell response of the transfected T cells including cytokine release assays, killing assays or cell proliferation assays. Examples 5, 6 and 7 herein provide details of a cytokine release assay, a killing assay and a cell proliferation respectively suitable for measure the level of T cell response.

One preferred embodiment of the present invention is provided by a method of taking a T cell-containing population of cells from a patient and transfecting said cells with a TCR having, in soluble form, a half-life for the interaction with the its cognate pMHC falling within the overlap range of preferred TCR half lives for transfection of CD4+ and CD8⁺ T cells. (9.6 seconds to 12 minutes) The transfected T cells obtained by this method will include both CD4⁺ and CD8⁺ T cells which are capable of being specifically activated by APCs presenting the cognate peptide-MHC for the transfected TCR.

T cells can be divided into “Killer” and “Helper” sub-types. Killer T cells are capable of directly killing infected or cancerous cells and are generally characterised by the expression of a heterodimeric co-receptor (CD8αβ) giving these cells a CD3⁺/CD8⁺ phenotype. Helper T cells are involved in initiating antibody-mediated responses to extracellular pathogens and these cells are characterised by the expression of a monomeric co-receptor (CD4) giving these cells a CD3⁺/CD4⁺ phenotype.

The TCR transfected T cells of the present invention are used to target abnormal cells presenting cancer or infection-specific pMHCs complexes. The pMHCs of cancerous cells may comprise peptides derived from proteins which are not expressed by corresponding non-cancerous cells and/or there may be abnormal levels of one or more normally occurring pMHC present of the surface of these cells. Pathogen (including but not limited to viral and bacterial) infection can also lead to characteristic changes in the pMHC profile of a subject. If the infectious agent actively enters the cells of the subject peptides derived from the agent are likely to be presented by Class I pMHCs on the surface of these cells. Additionally or alternatively, Class II pMHCs comprising peptides from the infective agent may be presented by uninfected antigen presenting cells which have taken up the infectious agents from the blood or lymph fluid of a subject. The presentation of such infection-specific Class II pMHC will facilitate an antibody-mediated immune response.

There are a number of known methods which can be used to produce and select mutated TCRs for use in the present invention. For example, mutated TCRs can be created by a number of methods, for example by the TCR phage method detailed in WO 2004/044004. Alternatively, or additionally, TCRs can be produced by hybridising the amino acid sequences of WT and mutated TCRs, and/or by pairing the alpha and beta chains of a plurality of TCRs with the same pMHC specificity.

The mutations required in order to produce TCRs from which can be selected those for use is the present invention may be made in any part of the TCR chains. For example, these mutations may be made in sequences within the variable regions of said TCRs, such as the CDR3, CDR2, CDR1 or HV4 regions therein.

TCRs for use in the present invention are defined by reference to their half lives (in soluble form) for their interactions with their cognate ligands. In order to measure the half-life of the interaction between a given soluble TCR and its cognate peptide-MHC soluble versions of the eventual transfectable TCR are produced. As will be known to those skilled in the art there are a number of TCR designs suitable for producing such soluble versions. Generally, these designs comprise TCR chains which have been truncated to remove the transmembrane regions thereof. WO 03/020763 describes the production and testing of soluble TCRs of a preferred design which utilises an introduced non-native disulfide interchain bond to facilitate the association of the truncated TCR chains. Details of other potentially suitable soluble TCR designs can be found in:
WO 99/60120 which described the production of non-disulfide linked truncated TCR chains which utilise heterologous leucine zippers fused to the C-termini thereof to facilitate chain association, and
WO 99/18129 which described the production of single-chain soluble TCRs comprising a TCRα variable domain covalently linked to a TCRβ variable domain via a peptide linker.

The measurement of the half-life of the interaction between a given soluble TCR and its cognate peptide-MHC ligand can be made by any of the known methods. A preferred method is the Surface Plasmon Resonance (Biacore) method of Example 2 herein. The data produced from the method described in Example 2 allows the following parameters for a given TCR/peptide-MHC interaction to be determined:

### Affinity (K_{D}) for the Interaction.

K_{D}=Off-rate(k_{off})/On-rate(kₒₙ)

### Half-Life (T_{1/2}) for the Interaction.

T_{1/2}=Ln 2/Off-rate(k_{off})

The T cells of the invention are transfected (either stably or transiently) with nucleic acids such that the latter are expressible in the cell. This will normal involve incorporating the nucleic acids into suitable expression vectors, of which many are known. For example, the T cells can be infected (“transduced”) with viruses or virus-derived proteins comprising nucleic acid or nucleic acids encoding TCRs. Alternatively, the T cells can be transfected with plasmids comprising nucleic acid or nucleic acids encoding TCRs, or the T cells can be incubated in the presence of “naked” nucleic acid or nucleic acids which encode TCRS s under conditions which allow the said nucleic acid or nucleic acid or nucleic acids to enter the T cells. Electroporation or lipofection are examples of methods typically used to enhance the entry of the “naked” or vector-borne TCR encoding nucleic acid or nucleic acids in to these T cells. The TCR-encoding nucleic acid or nucleic acids used in these transfection methods can be either DNA or RNA. The technology of recombinant DNA expression is well understood and described in many laboratory manuals and textbooks. (See, for example, Sambrook and Russell (2001) Molecular Cloning, a Laboratory Manual. 3^{rd} edition, ISBN 0-87969-576-5)

Examples 3 and 4 herein, provide suitable methods for carrying out the transfection of T cells with nucleic acid or nucleic acids encoding TCRs.

Although the nucleic acids of the invention are defined uniquely by their sequence information, they are intended to benefit from one or more of the following known general design considerations:

Relative abundance of transfer RNA (tRNA)—It is known that the cells of different species possess varying amounts of the tRNA molecules which each recognise the different codons which can be used to encode a given amino acid residue. Therefore, in general, it is preferable to use the codon recognised by the most abundance of these tRNA molecules. However, it may be advisable to use the selection of different codons encoding a given amino acid residues if these residue are encoded with high frequency within a sub-sequence of the nucleic acids, in order to prevent localised depletion of the most abundant tRNA species which may otherwise occur.

Removal of inverted repeat motifs—Such sequences introduce strong secondary structures, such as internal self hybridising “hair-pins”, into nucleic acids which may result in a slowing down of the translation process and a reduction of the level of expression.

Avoidance of other unwanted motifs—For example, the removal of inappropriate messenger RNA splice sites or polyadenylation signals, and undesirable restriction enzyme recognition DNA sequences.

Introduction of desired motifs—For example, translation initiation consensus signals (“Kozak” signals) 5′ of the ORF, and/or a strong translation termination codon, such as TAA immediately 3′ of the ORF, and/or efficient messenger RNA transcription termination signals.

Optimisation of nucleic acid GC content—The overall ratio of CG:AT bases in a nucleic acid can also influence the rate of transcription and/or translation of a nucleic acid encoding a given polypeptide.

Note however, it is not necessarily preferable to induce the fastest possible rate of transcription and/or translation. Overly high rates of either of these processes may result in the production of inappropriately folded and therefore inactive polypeptides. There are a number of companies that perform such gene codon optimisation as a service. GENEART AG, Germany is one such company.

The TCR-transfected T cells of the present invention can be used for the treatment of cancer including, but not limited to, the following cancers:

NY-ESO-positive, Telomerase-positive, Melan-A-positive, renal, ovarian, bowel, head & neck, testicular, lung, stomach, cervical, bladder, prostate or melanoma.

The TCR-transfected T cells of the present invention can be used for the treatment of infection including, but not limited to, the following infectious diseases:

HIV/AIDS, influenza and hepatitis.

Preferred features of each aspect of the invention are as for each of the other aspects mutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

FIG. 1a is the DNA sequence of the codon-optimised full-length wild-type 1G4 NY-ESO TCR alpha chain.

FIG. 1b is the DNA sequence of the codon-optimised full-length wild-type 1G4 NY-ESO TCR beta chain.

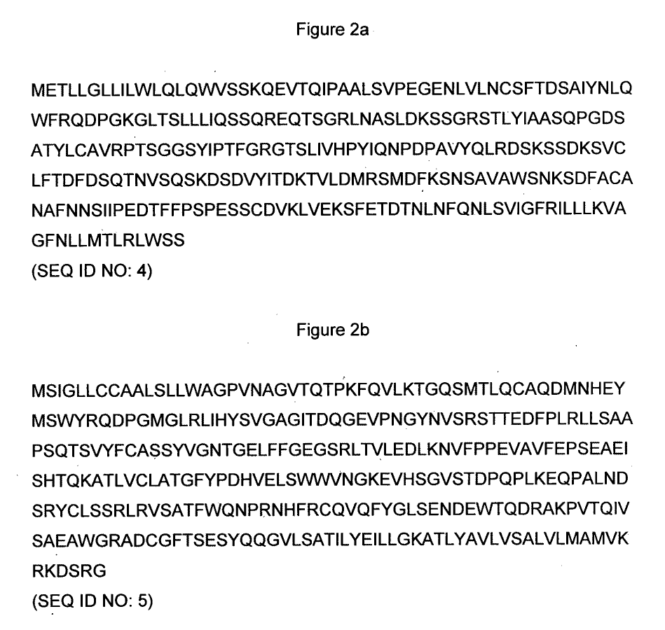

FIG. 2a is the amino acid sequence of the full-length 1G4 NY-ESO TCR wild-type alpha chain.

FIG. 2b is the amino acid sequence of the full-length 1G4 NY-ESO TCR wild-type beta chain.

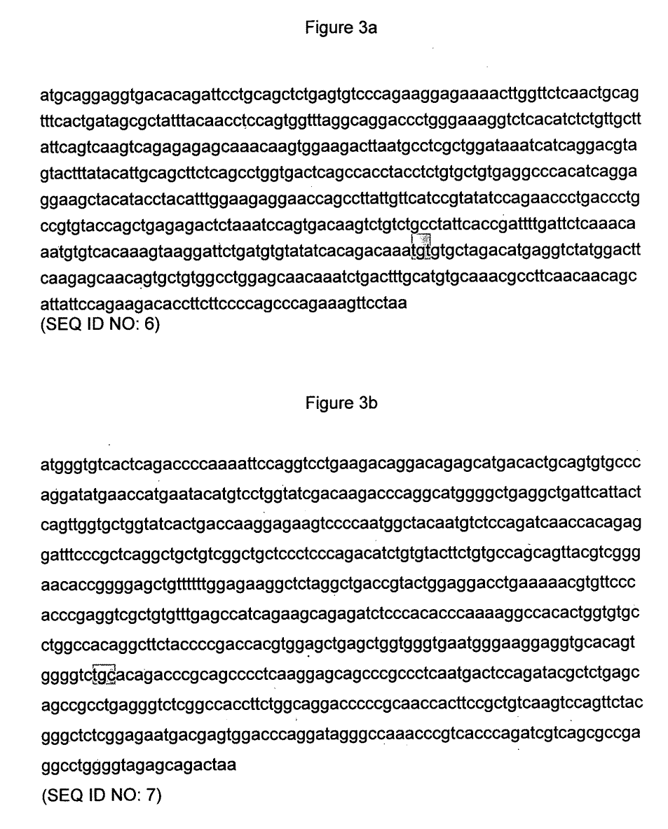

FIG. 3a is the DNA sequence of a soluble version of 1G4 NY-ESO TCR wild-type alpha chain including an introduced cysteine codon. The introduced cysteine codon is underlined.

FIG. 3b is the DNA sequence of is the DNA sequence of a soluble version of 1G4 NY-ESO TCR wild-type beta chain including an introduced cysteine codon. The introduced cysteine codon is underlined.

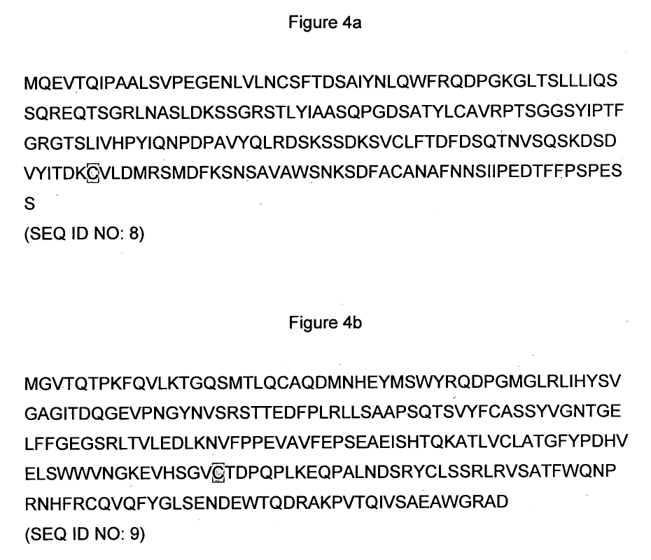

FIG. 4a is the amino acid sequence of a soluble version of 1G4 NY-ESO TCR wild-type alpha chain including an introduced cysteine codon. The introduced cysteine residue is highlighted.

FIG. 4b is the amino acid sequence of a soluble version of 1G4 NY-ESO TCR wild-type beta chain including an introduced cysteine codon. The introduced cysteine residue is highlighted.

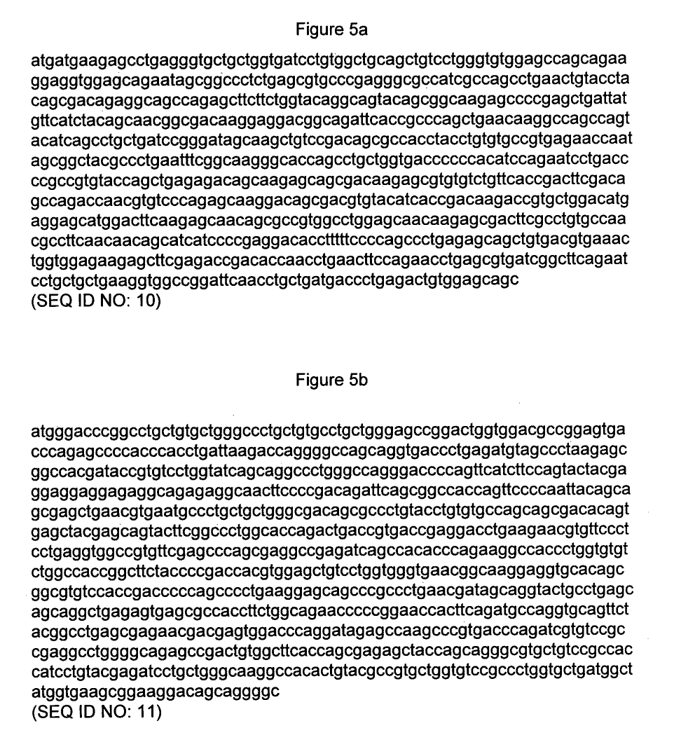

FIG. 5a is the DNA sequence of the codon-optimised full-length wild-type HIV Gag TCR alpha chain.

FIG. 5b is the DNA sequence of the codon-optimised full-length wild-type HIV Gag TCR beta chain.

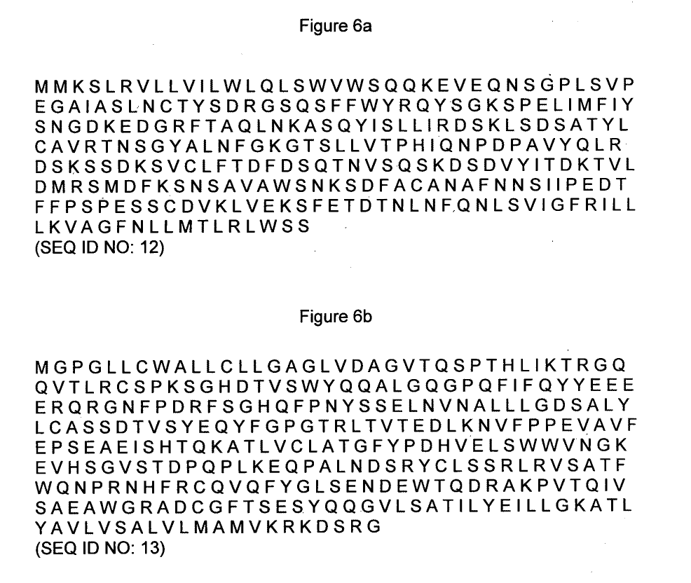

FIG. 6a is the amino acid sequence of the full-length wild-type HIV Gag TCR alpha chain.

FIG. 6b is the amino acid sequence of the full-length wild-type HIV TCR beta chain.

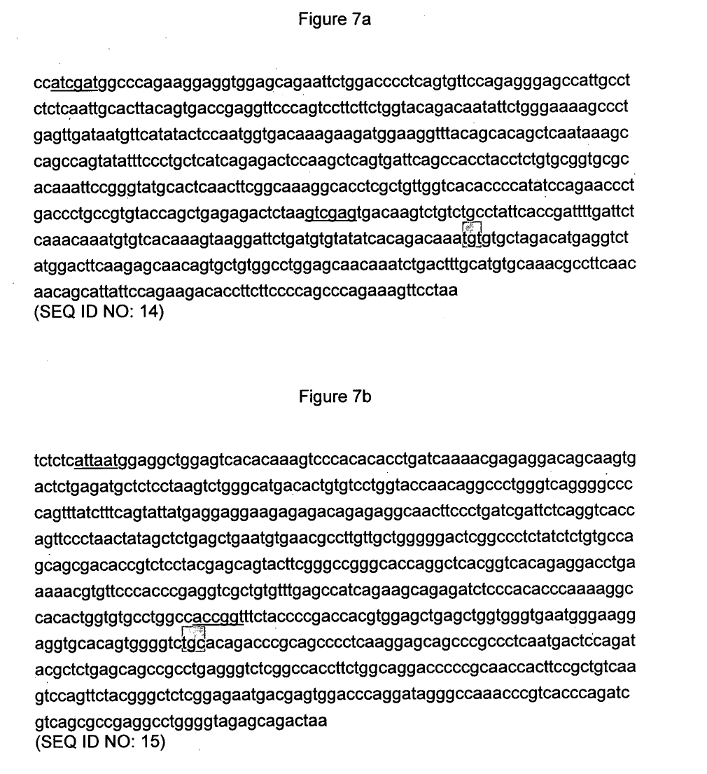

FIG. 7a is the DNA sequence of a soluble version of a wild-type HIV Gag TCR alpha chain including an introduced cysteine codon. The introduced cysteine codon is highlighted and the restriction enzyme recognition sites are underlined.

FIG. 7b is the DNA sequence of a soluble version of a wild-type HIV Gag TCR beta chain including an introduced cysteine codon. The introduced cysteine codon is highlighted and the restriction enzyme recognition sites are underlined.

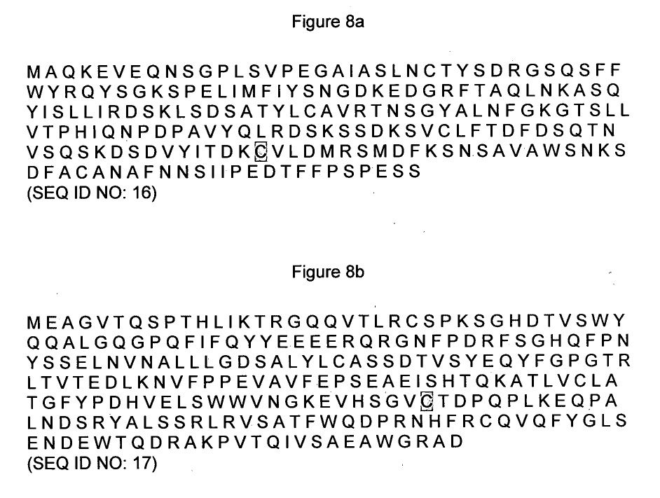

FIG. 8a is the amino acid sequence of a soluble version of the wild-type HIV Gag TCR alpha chain including an introduced cysteine residue. The introduced cysteine residue is highlighted.

FIG. 8b is the amino acid sequence of a soluble version of the wild-type HIV Gag TCR beta chain including an introduced cysteine residue. The introduced cysteine residue is highlighted.

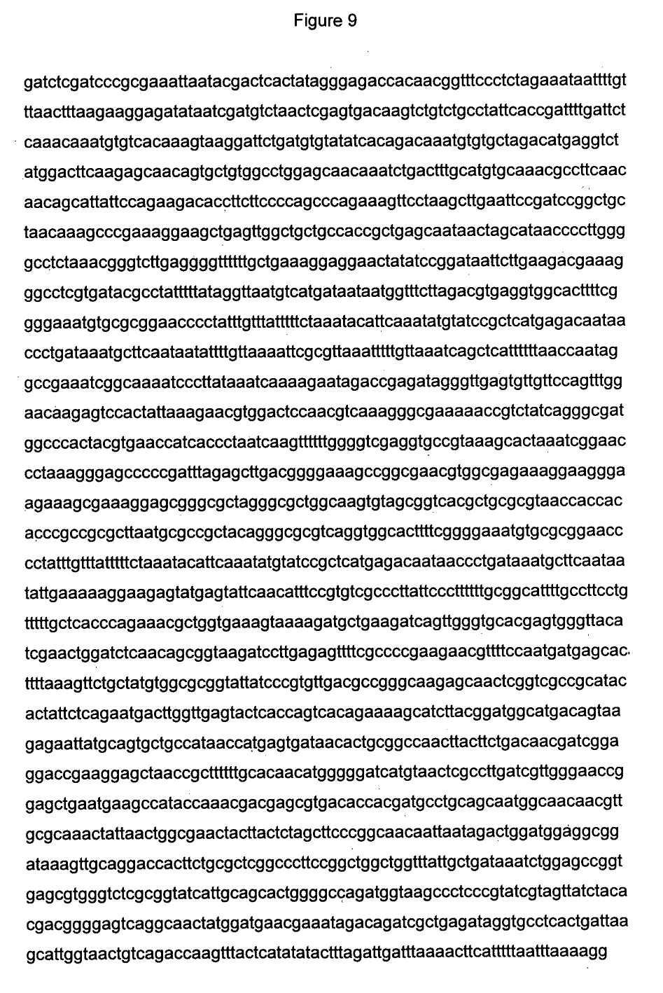

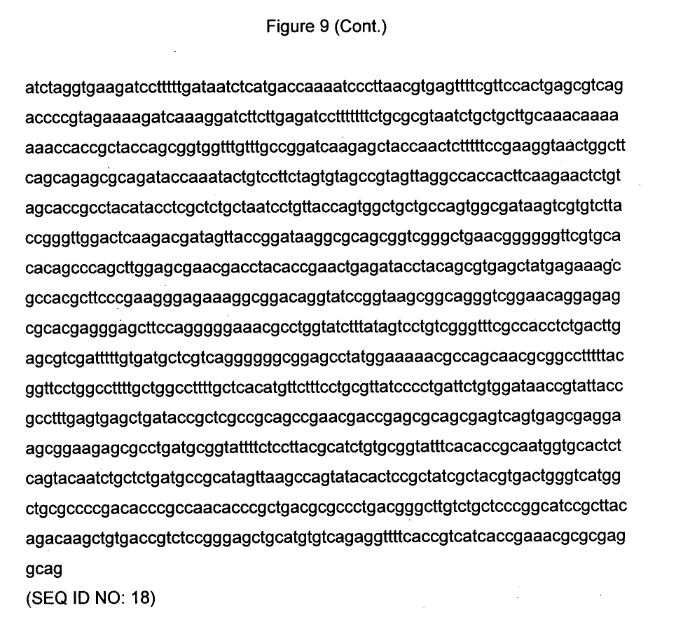

FIG. 9 is the DNA sequence of the pEX954 expression vector.

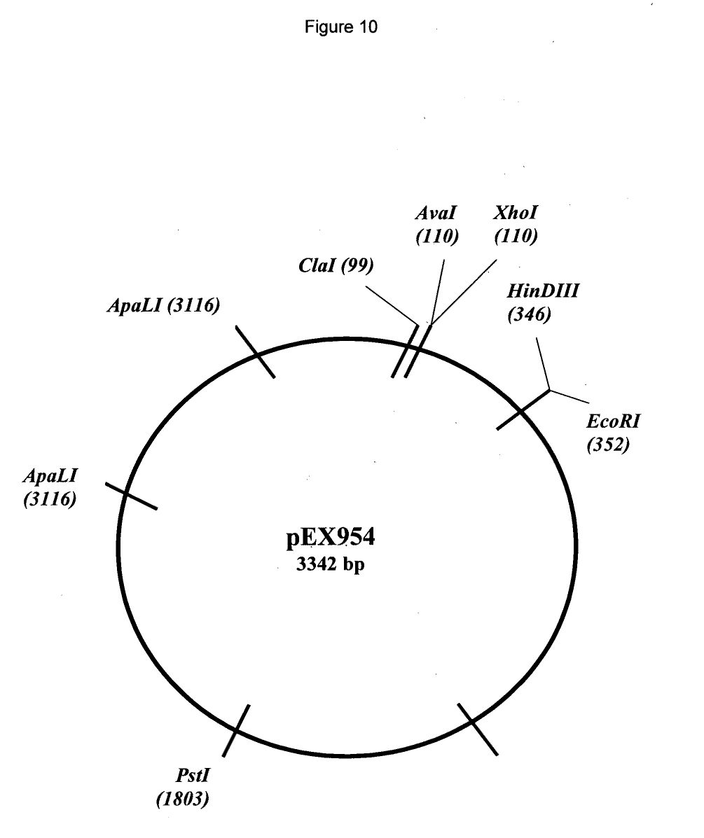

FIG. 10 is a plasmid map for the pEX954 expression vector.

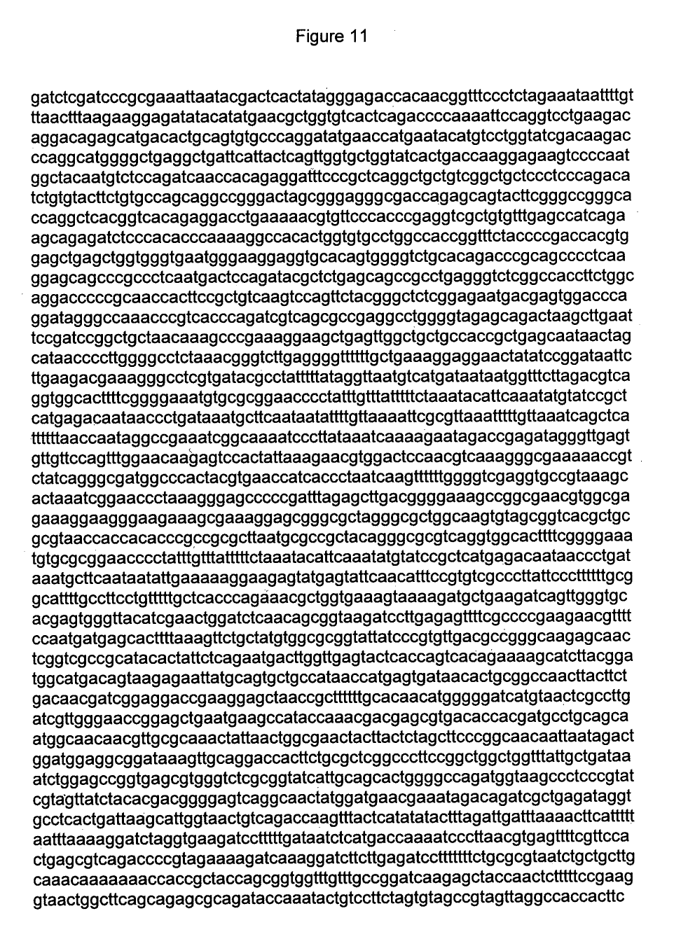

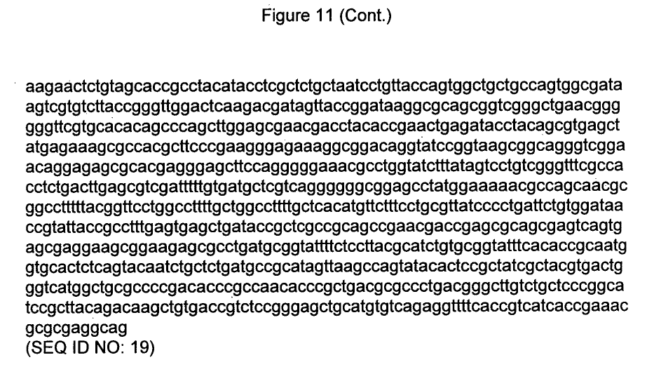

FIG. 11 is the DNA sequence of the pEX821 expression vector.

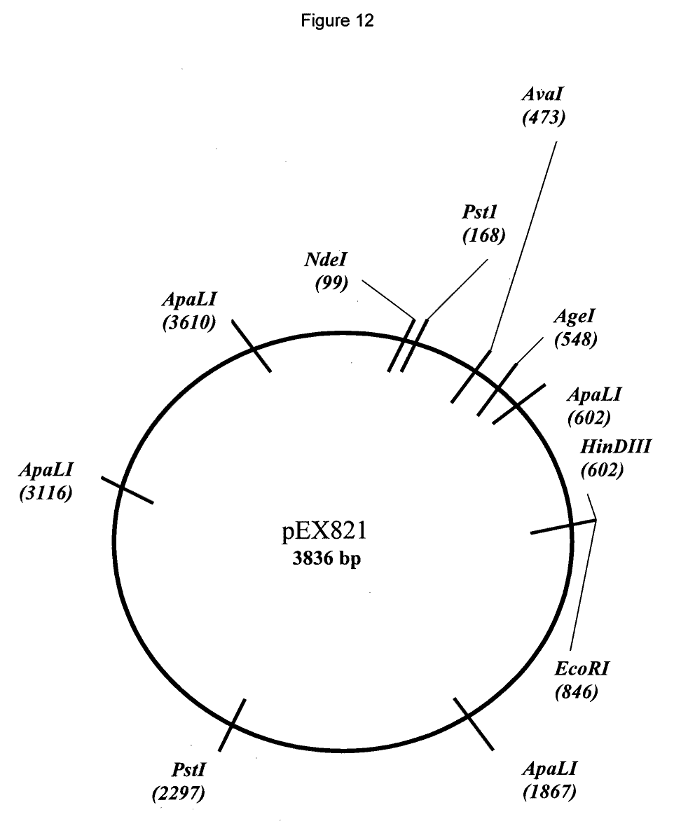

FIG. 12 is the plasmid map for the pEX821 expression vector.

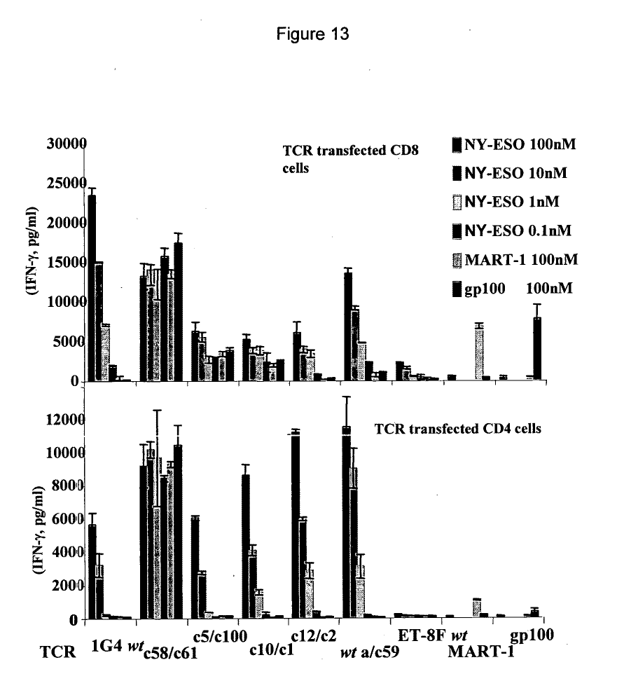

FIG. 13 is INF-γ release ELISA data showing activation of T cells transfected with nucleic acid encoding 1G4 NY-ESO TCRs.

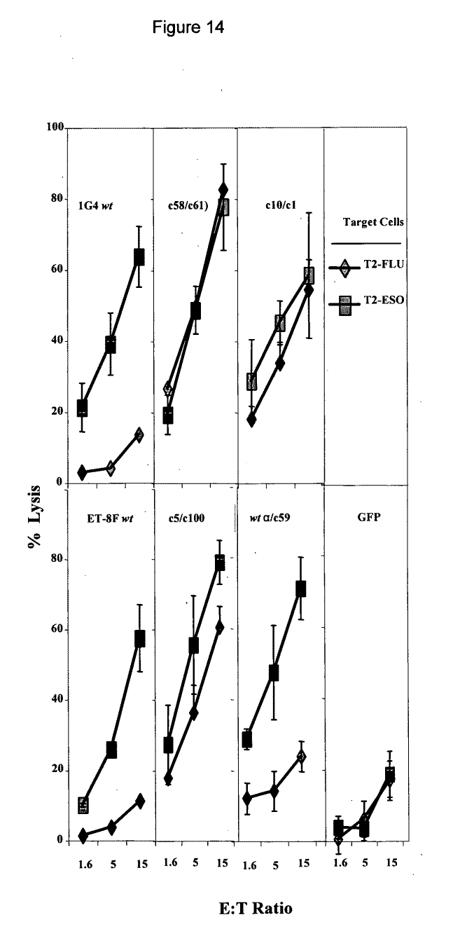

FIG. 14 is Chromium release data showing killing of APCs by CD8⁺ T cells transfected with nucleic acid encoding 1G4 NY-ESO TCRs.

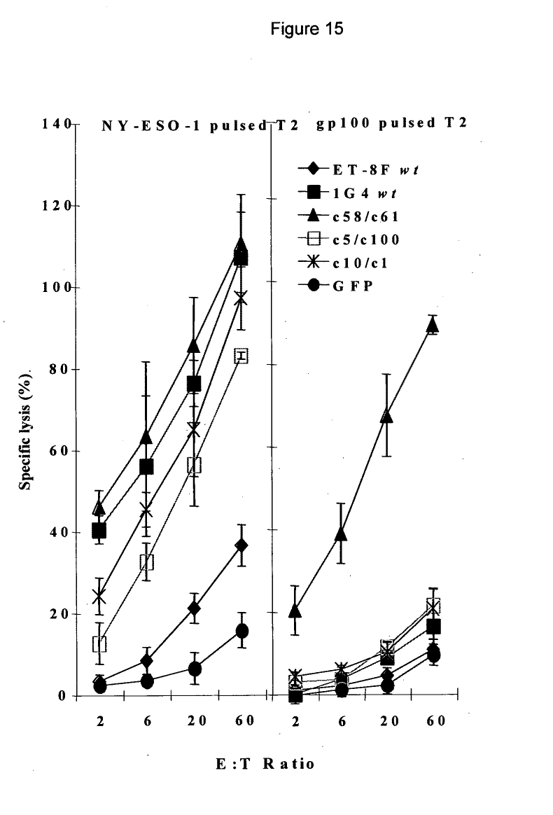

FIG. 15 is Chromium release data showing killing of APCs by CD4⁺ T cells transfected with nucleic acid encoding 1G4 NY-ESO TCRs.

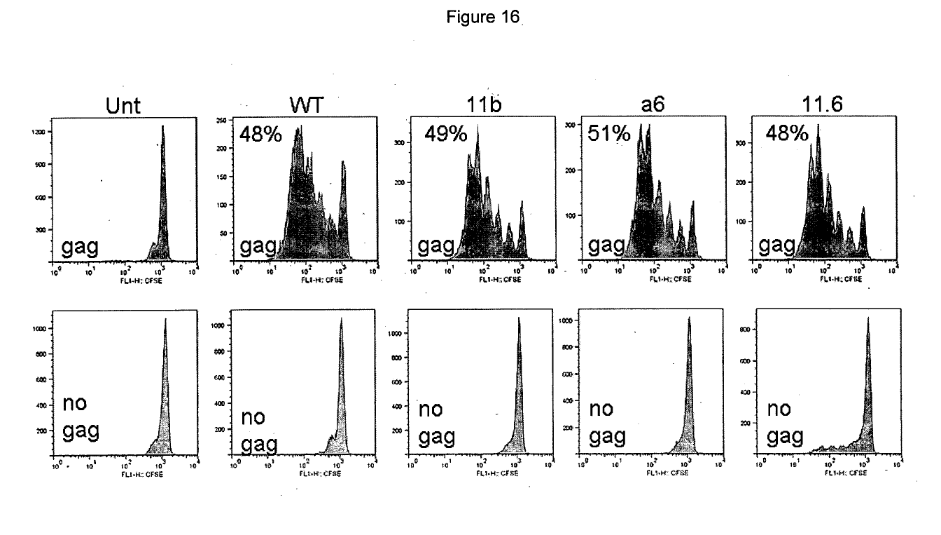

FIG. 16 is FACS data showing proliferation of CD8⁺ T cells transfected with nucleic acid encoding HIV Gag TCRs.

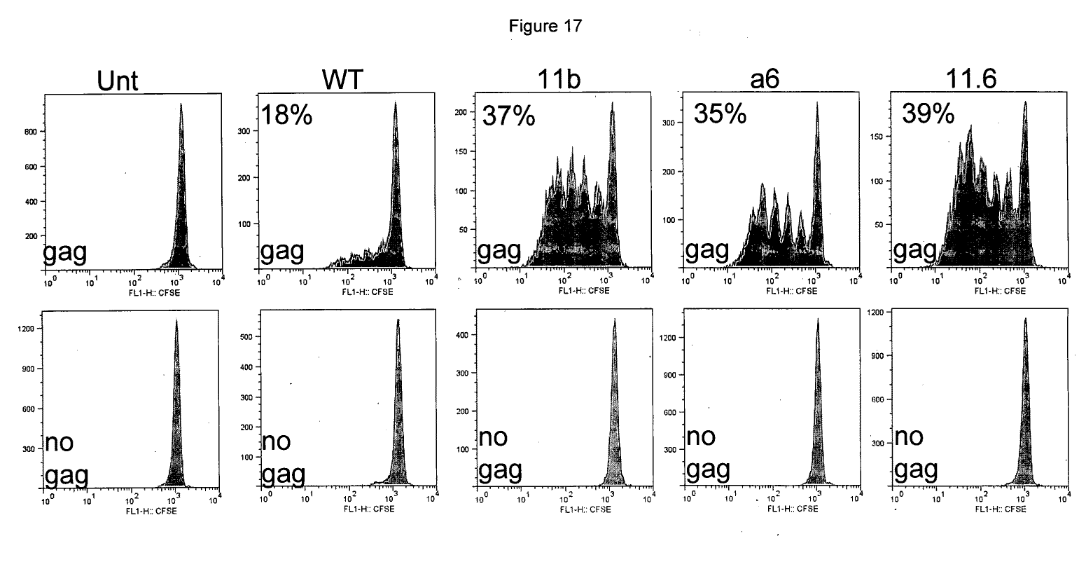

FIG. 17 is FACS data showing proliferation of CD4⁺ T cells transfected with nucleic acid encoding HIV Gag TCRs.

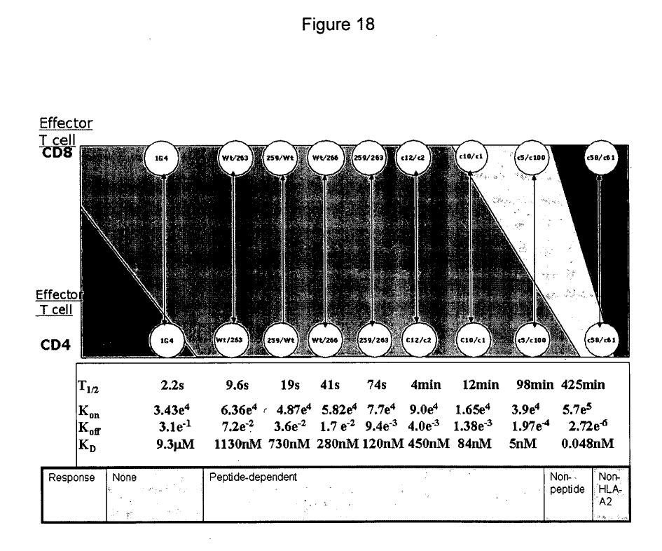

FIG. 18 is a diagram plotting the observed responses of T cells transfected with 1G4 NY-ESO TCRs against the Biacore-determined half-life of the corresponding soluble TCR.

### EXAMPLE 1

### Production of Soluble Disulfide Linked Versions of 1G4 NY-ESO and HIV Gag TCRs

FIGS. 3a and 3b provide the DNA sequences of the TCR alpha and beta chains of a soluble version of the wild-type 1G4 NY-ESO TCR. Each of these DNA sequences contains an introduced cysteine codon which is underlined.

FIGS. 7a and 7b provide the DNA sequences of the TCR alpha and beta chains of a soluble version of the wild-type HIV Gag TCR. Each of these DNA sequences contains an introduced cysteine codon which is underlined.

These DNA sequences can be synthesis de-novo by a number of contract research companies, for example GENEART AG (Germany).

Restriction enzyme recognition sites can be added to these DNA sequences in order to facilitate ligation of these DNA sequences into expression plasmids. pGMT7-based expression plasmids, which contain the T7 promoter for high level expression in E. coli strain BL21-DE3(pLysS (Pan et al., Biotechniques (2000) 29 (6): 1234-8)) are appropriate expression vectors.

ClaI and SalII restriction enzyme recognition sites were introduced into the above TCR alpha chain DNA sequences and these were ligated into pEX954 cut with ClaI and XhoI. (See FIGS. 9 and 10 respectively for the DNA sequence and plasmid map of the pEX954 vector).

AseI and AgeI restriction enzyme recognition sites were introduced into the above TCR beta chain DNA sequences and these were ligated into pEX821 cut with NdeI/AgeI. (See FIGS. 11 and 12 respectively for the DNA sequence and plasmid map of the pEX821 vector).

Restriction Enzyme Recognition Sites as Introduced into DNA Encoding the TCR Chains

| | |
|---|---|
| ClaI- | ATCGAT |
| SalII- | GTCGAC |
| AseI- | ATTAAT |
| AgeI- | ACCGGT |

### Ligation

The cut TCR alpha and beta chain DNA and cut vector were ligated using a rapid DNA ligation kit (Roche) following the manufacturers instructions.

Ligated plasmids were transformed into competent E. coli strain XL1-blue cells and plated out on LB/agar plates containing 100 mg/ml ampicillin. Following incubation overnight at 37° C., single colonies were picked and grown in 10 ml LB containing 100 μg/ml ampicillin overnight at 37° C. with shaking. Cloned plasmids were purified using a Miniprep kit (Qiagen) and the insert was sequenced using an automated DNA sequencer (Lark Technologies).

FIGS. 4a and 4b respectively are the soluble disulfide linked wild-type 1G4 TCR α and β chain amino acid sequences produced from the DNA sequences of FIGS. 3a and 3b

FIGS. 8a and 8b respectively are the soluble disulfide linked wild-type HIV gag TCR α and β chain amino acid sequences produced from the DNA sequences of FIGS. 7a and 7b

The above methods can be used to produce soluble disulfide linked versions of mutated IG4 NY-ESO TCRs or HIV Gag TCRs. Suitable mutated TCRs can be identified by a number of methods, for example by the TCR phage display method detailed in WO 2004/044004.

Soluble versions of these mutated TCRs are produced by altering the DNA sequence encoding the corresponding wild-type or wild-type TCR chain to produce the required mutations.

### EXAMPLE 2

### Biacore Surface Plasmon Resonance Characterisation of sTCR Binding to Specific pMHC

A surface plasmon resonance biosensor (Biacore 3000™) was used to analyse the binding of a sTCR to its peptide-MHC ligand. This was facilitated by producing single pMHC complexes (described below) which were immobilised to a streptavidin-coated binding surface in a semi-oriented fashion, allowing efficient testing of the binding of a soluble T-cell receptor to up to four different pMHC (immobilised on separate flow cells) simultaneously. Manual injection of HLA complex allows the precise level of immobilised class I MHC molecules to be manipulated easily.

Biotinylated class I HLA-A*0201 molecules were refolded in vitro from bacterially-expressed inclusion bodies containing the constituent subunit proteins and synthetic epitope peptide, followed by purification and in vitro enzymatic biotinylation (O'Callaghan et al. (1999) Anal. Biochem. 266: 9-15). HLA-A*0201-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The MHC light-chain (β2-microglobulin) was also expressed as inclusion bodies in E. coli from an appropriate construct, at a level of ˜500 mg/litre bacterial culture.

E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2 microglobulin into 0.4 M L-Arginine-HCl, 100 mM Tris pH 8.1, 3.7 mM cystamine, 6.6 mM cysteamine, 4 mg/ml of the cognate epitope peptide required to be loaded by the HLA-A*0201 molecule, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μm cellulose acetate filter and loaded onto a POROS 50HQ anion exchange column (8 ml bed volume). Protein was eluted with a linear 0-500 mM NaCl gradient. HLA-A*0201-peptide complex eluted at approximately 250 mM NaCl, and peak fractions were collected, a cocktail of protease inhibitors (Calbiochem) was added and the fractions were chilled on ice.

Biotinylation tagged pMHC molecules were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 mM biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture was then allowed to incubate at room temperature overnight.

The biotinylated pHLA-A*0201 molecules were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated pHLA-A*0201 molecules eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated pHLA-A*0201 molecules were stored frozen at −20° C. Streptavidin was immobilised by standard amine coupling methods.

Such immobilised complexes are capable of binding both T-cell receptors and the coreceptor CD8αα, both of which may be injected in the soluble phase. Specific binding of TCR is obtained even at low concentrations (at least 40 μg/ml), implying the TCR is relatively stable. The pMHC binding properties of soluble TCR (sTCR) are observed to be qualitatively and quantitatively similar if sTCR is used either in the soluble or immobilised phase. This is an important control for partial activity of soluble species and also suggests that biotinylated pMHC complexes are biologically as active as non-biotinylated complexes.

The interactions between sTCR containing a novel inter-chain bond and its ligand/MHC complex or an irrelevant HLA-peptide combination, the production of which is described above, were analysed on a Biacore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The probe flow cells were prepared by immobilising the pMHC complexes in flow cells via biotin-tag binding. The assay was then performed by passing sTCR over the surfaces of the different flow cells at a constant flow rate, measuring the SPR response in doing so.

### To Measure Equilibrium Binding Constant

Serial dilutions of WT sTCR were prepared and injected at constant flow rate of 5 μl min-1 over two different flow cells; one coated with ˜1000 RU of the cognate peptide-HLA-A*0201 complex, the second coated with ˜1000 RU of non-specific peptide-HLA-A*0201 complex. Response was normalised for each concentration using the measurement from the control cell. Normalised data response was plotted versus concentration of TCR sample and fitted to a hyperbola in order to calculate the equilibrium binding constant, K_{D}. (Price & Dwek, Principles and Problems in Physical Chemistry for Biochemists (2^{nd} Edition) 1979, Clarendon Press, Oxford).

### To Measure Kinetic Parameters

For high affinity TCRs K_{D} was determined by experimentally measuring the dissociation rate constant, kd, and the association rate constant, ka. The equilibrium constant K_{D} was calculated as kd/ka.

TCR was injected over two different cells one coated with ˜300 RU of the cognate peptide-HLA-A2*0201 complex, the second coated with ˜300 RU of non-specific peptide-HLA-A*0201 complex. Flow rate was set at 50 μl/min. Typically 250 μl of TCR at ˜3 μM concentration was injected. Buffer was then flowed over until the response had returned to baseline. Kinetic parameters were calculated using Biaevaluation software. The dissociation phase was also fitted to a single exponential decay equation enabling calculation of half-life.

### Results

The following tables summarise the Biacore determined affinity (K_{D}) and half-lives for the interaction between soluble disulfide-linked versions of WT 1G4 NY-ESO and WT HIV GAG TCRs and several mutants thereof, identified, made and tested by the above procedures, and their cognate peptide-MHC complexes.

### 1G4 NY-ESO-Based TCRs

| | | |
|---|---|---|
| TCR | | |
| Identifier | Half-live (T½) | Affinity (K_{D}) |

| | | | | |
|---|---|---|---|---|
| Wild-type (WT) | 2.2 | seconds | 9.3 | μM |
| wt/263 | 9.6 | seconds | 1.13 | μM |
| 259/wt | 19 | seconds | 730 | nM |
| wt/266 | 41 | seconds | 280 | nM |
| 259/263 | 74 | seconds | 120 | nM |
| c12/c2 | 4 | minutes | 450 | nM |
| c10/c1 | 12 | minutes | 84 | nM |
| C5/c100 | 98 | minutes | 5 | nM |
| c58/c61 | 425 | minutes | 0.048 | nM |

### HIV Gag-Based TCRs

| | | |
|---|---|---|
| TCR | | |
| Identifier | Half-live (T½) | Affinity (K_{D}) |

| | | | |
|---|---|---|---|
| Wild-type (WT) | 31 | seconds | 165 nM |
| c11/wt | 7.7 | minutes | 8.7 nM |
| wt/c6 | 12 | minutes | 4.9 nM |
| c11/c6 | 162 | minutes | 365 pM |

### EXAMPLE 3

### Production of Codon Optimised DNA and RNA Encoding Full Length 1G4 NY-ESO and HIV Gag TCRs

FIGS. 1a and 1b provide the DNA sequences of the TCR alpha and beta chains of codon-optimised full-length wild-type 1G4 NY-ESO TCR.

FIGS. 5a and 5b provide the DNA sequences of the TCR alpha and beta chains of codon-optimised full-length wild-type HIV Gag TCR.

These DNA sequences can be synthesis de-novo by a number of contract research companies, for example GENEART AG (Germany).

Restriction enzyme recognition sites can be added to these DNA sequences in order to facilitate ligation of these DNA sequences into appropriate gene expression vectors. Examples of such appropriate gene expression vectors include retroviral vectors such as derivatives of the MSCV-based splice-gag vector (pMSGV) which is described in Hughes et al., (2005) Hum Gene Ther. 16: 457-472. Retroviral packaging and T cell transduction can then be carried out according to Zhao et al. (2005) J Immunol. 174: 4415-4423. Alternatively, TCRs genes can be evaluated by transfection of T cells using in-vitro transcribed (IVT) RNA corresponding to the TCR DNA sequences provided herein. See Zhao et al. (2006) Mol. Ther. 13: 151-159 for details of the methods required. Briefly, PCR primers were designed to amplify plasmid-encoded TCR genes and introduce a T7 promoter at the 5′ end and a polyA tract at the 3′ the genes for the alpha and beta TCR chains respectively. By using these purified PCR products as templates, RNA was generated via in vitro transcription.

FIGS. 2a and 2b respectively are the full-length wild-type 1G4 TCR α and β chain amino acid sequences produced from the DNA sequences of FIGS. 1a and 1b

FIGS. 6a and 6b respectively are the full-length wild-type HIV gag TCR α and β chain amino acid sequences produced from the DNA sequences of FIGS. 5a and 5b

Mutated IG4 NY-ESO TCRs or HIV Gag TCRs can be identified by a number of methods, for example by the TCR phage display method detailed in WO 2004/044004. The mutations thus identified can be introduced into the full length gene optimised DNA or RNA sequences encoding the wild-type or wild-type TCR chains.

### EXAMPLE 4

### Electroporation of T Cells with IVT RNA Encoding IG4 NY-ESO TCRs

Electroporation of anti-CD3 antibody (OKT3) stimulated human PBLs and cell lines with IVT RNA encoding the 1G4 NY-ESO TCRs was conducted as described in Zhao et al. (2006) Mol. Ther. 13: 151-159.

The RNA encoding the WT NY-ESO TCR alpha chain sequence corresponds to the DNA sequence provided in FIG. 1a. The RNA encoding the WT NY-ESO TCR beta chain sequence corresponds to the DNA sequence provided in FIG. 1b.

### Results

The 1G4 TCR IVT RNA transfection efficiency levels obtained were between 30% and 75%. (FACS analysis using PE-labelled streptavidin cognate peptide-HLA-A*0201 tetramer staining was used to determine these values).

### EXAMPLE 5

### Cytokine Release ELISA Assays of CD8⁺ and CD4⁺ T Cells Transfected with 1G4 NY-ESO TCRs

Peripheral Blood Lymphocyte (PBL) T cell cultures were transfected with IVT RNA encoding the following 1G4NY-ESO TCRs:

WT, wt/263, 259/wt, wt/266, 259/263, c12/c2. c10/c1, c5/c100 and c58/c61.

These TCRs have Biacore-determined monomer half-lives of 2.2 seconds, 9.6 seconds, 19 seconds, 41 seconds, 74 seconds, 4 minutes, 12 minutes, 98 minutes and 425 minutes respectively.

These transfected T cells were tested for reactivity in cytokine release assays using a commercially available ELISA kit (IFN-γ; Endogen, Cambridge, Mass.). T2 APCs were pulsed with cognate or non-cognate peptides in R/10 medium for 2 hrs at 37° C., followed by washing (three times) before initiation of co-cultures. The TCR transfected T cells and responder APC cells were co-cultured for 24 h. Cytokine secretion was measured in culture supernatants diluted to be in the linear range of the assay.

### Results

The illustrative IFN-γ release data presented in FIG. 13 shows that CD4⁺ T cells transfected with the wild-type (WT), c5/c100, c10/c1 and c12c2 mutant 1G4 NY-ESO TCRs respond to APCs in a cognate antigen specific manner. However, CD4⁺ T cells transfected with the wild-type (WT) 1G4 NY-ESO TCRs only respond significantly to the cognate peptide when the APCs are pulsed at high (non-physiologically-relevant) peptide levels. (10 nM or higher) Further IFN-γ release data (not shown) demonstrates that CD4⁺ T cells transfected with the wt/263, 269/wt, wt/266 and 259/263 mutated 1G4 NY-ESO TCRs respond to APCs in a cognate antigen specific manner.

The data presented in FIG. 13 also shows that CD4⁺ T cells transfected with the c58/c61 mutant 1G4 NY-ESO TCRs respond to APCs in a non-cognate antigen specific manner.

The data on IFN-γ release from CD8⁺ T cells transfected with the wild-type (WT) and c12c2 mutant 1G4 NY-ESO TCRs demonstrates that these transfected T cells respond to APCs in a cognate antigen specific manner. The data on IFN-γ gamma release from CD8+ T cells transfected with the c58/c61, c5/c100, and c10c1 mutant 1G4 NY-ESO TCRs demonstrates that these transfected T cells respond to APCs in a non-cognate antigen specific manner. Further IFN-γ release data (not shown) demonstrates that CD8⁺ T cells transfected with the wt/263, 269/wt, wt/266 and 259/263 mutated 1G4 NY-ESO TCRs respond to APCs in a cognate antigen specific manner.

### Conclusions

These results demonstrate that CD8⁺ T cells transduced to express the wild-type (WT) wt/263, 259/wt, wt/266, 259/263 and c12c2 mutant 1G4 TCRs (which have Biacore determined monomer half-lives of 2.2 seconds, 9.6 seconds, 19 seconds, 41 seconds, 74 seconds and 4 minutes respectively) respond specifically to APCs presenting a physiologically relevant level of the cognate antigen. CD8⁺ T cells transfected with the c58/c61, c5/c100 and c10/c1 mutant 1G4 NY-ESO TCRs (which have Biacore determined monomer half-lives of 425 minutes, 98 minutes, 12 minutes respectively) also respond to APCs presenting non-cognate antigen.

These results demonstrate that the upper limit of TCR half-life for “physiologically-relevant” cognate antigen-specific T cells responses in CD8⁺ T cells lies between 4 and 12 minutes.

These results demonstrate that CD4⁺ T cells transduced to express wt/263, 259/wt, wt/266, 259/263, c12/c2, c10/c1, c5/c100, mutant 1G4 TCRs (which have Biacore determined monomer half-lives of 9.6 seconds, 19 seconds, 41 seconds, 74 seconds, 4 minutes, 12 minutes and 98 minutes respectively) respond to APCs in a “physiologically relevant” cognate antigen-specific manner. CD4⁺ T cells transfected with the c58/c61 mutant 1G4 NY-ESO TCRs (which has a Biacore determined monomer half-life of 425 minutes) also responded to APCs not presenting the cognate antigen.

These results demonstrate that the lower limit of TCR half-life for “physiologically-relevant” cognate antigen-specific T cells responses in CD4⁺ T cells lies between 2.2 and 9.6 seconds.

These results demonstrate that the upper limit of TCR half-life for cognate antigen-specific T cells responses in CD4⁺ T cells lies between 98 and 425 minutes.

### EXAMPLE 6

### Chromium Release 1G4 NY-ESO TCR Transfected T Cell Killing Assay

The ability of T cells transfected with WT and mutant 1G4 NY-ESO TCRs to lyse antigen-specific peptide-pulsed target cells was measured using a chromium (⁵¹Cr) release assay. Briefly, 1×10⁶ target APCs were labeled for 1 h at 37° C. with 200 μCi of ⁵¹Cr sodium chromate (GE Healthcare, Piscataway, N.J.). Labeled target cells (5×10³) were incubated with effector cells at the ratios indicated in the text for 4 h at 37° C. in 0.2 ml of R/10 medium. Harvested supernatants were counted using a Wallac 1470 Wizard gamma counter (PerkinElmer, Wellesley, Mass.). Total and spontaneous ⁵¹Cr release was determined by incubating 5×10³ labeled target cells in either 2% SDS or R/10 medium for 4 h at 37° C. Each data point was determined as an average of quadruplicate wells. The percent specific lysis was calculated as follows: % specific lysis=((specific ⁵¹Cr release−spontaneous ⁵¹Cr release)/(total ⁵¹Cr release−spontaneous ⁵¹Cr release))×100.

### Results

The killing data presented in FIG. 14 shows that CD8⁺ T cells transfected with the wild-type (WT) and wt/c59 mutant 1G4 NY-ESO TCRs respond to peptide pulsed APCs in a cognate antigen specific manner. T cells transfected with the c10/c1, c5/c100 and c58/c61 mutant 1G4 NY-ESO TCRs responded to peptide pulsed APCs in a non-cognate antigen specific manner.

The killing data presented in FIG. 15 shows that CD4⁺ T cells transfected with the wild-type (WT) and c10/c1c5/c100 and wt/c59 mutant 1G4 NY-ESO TCRs respond to peptide pulsed APCs in a cognate antigen specific manner. T cells transfected with the c58/c61 mutant 1G4 NY-ESO TCRs responded to peptide pulsed APCs in a non-cognate antigen specific manner.

### Conclusions

These peptide-pulsed target APC lysis data broadly accord to that obtained by the IFN-γ release ELISA assays detailed in Example 5 above.

### EXAMPLE 7

### Dye Depletion HIV Gag TCR Transduced T Cells Proliferation Assay

T cells were transduced with DNA encoding the wild-type and mutated HIV Gag TCRs using methods substantially as described in Parry et al., (2003) J. Immunol 171: 166-174. Briefly, TCR α chain and TCR β chain encoding DNA sequences were inserted together into a Lentiviral expression vector. This vector contains DNA encoding both the TCR α chain and β chain as a single open reading frame with the in-frame Foot and Mouth Disease Virus (FMDV) 2A cleavage factor amino acid sequence (LLNFDLLKLAGDVESNPG (SEQ ID NO: 1)) separating the TCR chains. (de Felipe et al., (2004) Genet Vaccines Ther 2 (1): 13) On mRNA translation the TCR α chain is produced with the 2A peptide sequence at its C-terminus and the TCR β chain is produced as a separate polypeptide.

The ability of CD8⁺ and CD4⁺ T cells transduced with HIV Gag TCRs to proliferate in the presence of either untransfected K562 APCs or K562 APCs transfected to express the cognate Gag HIV epitope was assessed. This was carried by FACs analysis of the transduced T cells which had been stained with carboxyfluorescein diactetate succinimidyl ester (CFSE). CFSE is a dye which is can passively diffuse into cells and then reacts with intracellular amines to form fluorescent conjugates which are retained within the cell. Proliferation of the stained T cells can be monitored by a reduction in the average fluorescent of the T cells which occurs as the cells divide and the dye is then diluted between the parent and daughter cells. FIGS. 16 and 17 provide FACs data from HIV Gag TCR transduced CD8+ T cells and CD4+ T cells respectively.

### Results

FIG. 16 shows that CD8⁺ T cells transduced to express the wild-type HIV Gag TCR (WT) and CD8⁺ T cells transduced to express the mutated c11/wt, wt/c6 and c11/c6 HIV Gag TCR proliferate in the present of K562 APCs expressing the cognate HIV Gag epitope. T cells transduced to express the c11/c6 mutated HIV Gag TCR also proliferate in the presence of K562. APCs which do not express the cognate epitope.

FIG. 17 shows that CD4⁺ T cells transduced to express the wild-type HIV Gag TCR (WT) and CD4⁺ T cells transduced to express the mutated c11/wt, wt/c6 and c11/c6 HIV Gag TCR proliferate only in the presence of K562 APCs expressing the cognate HIV Gag epitope.

### CONCLUSION

These results demonstrate that CD8⁺ T cells transduced to express the WT, c11/wt and wt/c6 mutant HIV Gag TCRs (which have Biacore determined monomer half-lives of 31 seconds, 7.7 minutes and 12 minutes respectively) respond to APCs in a cognate antigen-specific manner. In contrast, CD8⁺ T cells transduced to express the c11/c6 mutant HIV Gag TCR (which has a Biacore determined monomer half-life of 162 minutes) responds weakly to APCs in a non-cognate antigen-specific manner.

These results demonstrate that the upper limit of TCR half-life for cognate antigen-specific T cell responses in CD8⁺ T cells lies between 12 and 162 minutes. This value is in broad agreement with the upper limit of TCR half-life for cognate antigen-specific T cell responses in CD8⁺ T cells of between 4 and 12 minutes determined by the IFN-γ release ELISA assay carried on T cells transfected with 1G4 NY-ESO TCRs. In combination, these two data-sets indicate that this half-life upper limit is approximately 12 minutes within the limits of the differing experimental methodologies.

These results demonstrate that the lower limit of TCR half-life for cognate antigen-specific T cell responses in CD4⁺ T cells is lower than 31 seconds. This value is in agreement with the lower limit of TCR half-life for cognate antigen-specific T cell responses in CD4⁺ T cells of between 2.2 and 9.6 seconds determined by the IFN-γ release ELISA assay carried on T cells transfected with 1G4 NY-ESO TCRs.

These results also demonstrate that transduction of CD4⁺ T cells with mutated TCRs having half-lives longer than the corresponding WT TCR elicit an antigen-specific cell proliferation which is enhanced compared to that seen with CD4⁺ T cells transduced with the corresponding WT TCR. In contrast to the results obtained with transduced CD8⁺ T cells, CD4⁺ T cells transduced to express TCRs with half-lives of up to and including 162 minutes retain antigen specificity. Both these observations are in accordance with the IFN-γ release ELISA data obtained for CD4+ T cells transfected with 1G4 NY-ESO TCRs.

Finally, it should be noted that the Biacore-determined monomer half-life of a given TCR for its cognate pMHC has been established as the key interaction criterion predictive of cell function in T cells transfected with said TCRs. FIG. 18 provides a diagrammatic summary of the effect of TCR half-life on the nature of T cell function observed for CD4+ and CD8+ T cells expressing said TCRs. To illustrate this further CD8+ T cells transfected with the c11/wt mutated HIV Gag TCR which has a Biacore-determined monomer half-life of 7.7 minutes would be expected to function in a cognate antigen specific manner and this is the case. However, the Biacore-determined monomer affinity (K_{D}) of the c11/wt HIV Gag TCR is 8.7 nM which is close to the determined affinity of the c5/c100 1G4 NY-ESO TCR which when transfected into CD8+ T cells leads to non-cognate antigen-specific T cell function.

1. A method of treatment of a disease selected from cancer and infection comprising administering to a subject suffering such disease a plurality of TCR-transfected T cells which are specifically activated by cells presenting a given pMHC characteristic of such disease, at least some of the TCRs presented by each of said T cells having, in soluble form, a half-life for the interaction with the said pMHC which is either
slower than that of the known corresponding wild type soluble TCR, or
in the case where no corresponding wild-type TCR is known:
(a) 1 second or slower in the case of a class I-restricted TCR transfected into a CD8⁺ T cell, or
(b) 9.6 seconds or slower in the case of a class I-restricted TCR transfected into a CD4⁺ T cell, or in the case of class II-restricted TCR transfected into a CD8⁺ T cell, or
(c) 0.9 seconds or slower in the case of a class II-restricted TCR transfected into a CD4⁺ T cell.

2. (canceled)

3. The method of claim 1 wherein at least some of the TCRs presented by said transfected T cells have, in soluble form, a half-life for the interaction with the said pMHC which is either
(a) 12 minutes or faster in the case of a class I-restricted TCR transfected into a CD8⁺ T cell, or
(b) faster than 425 minutes in the case of a class I-restricted TCR transfected into a CD4⁺ T cell, or in the case of class II-restricted TCR transfected into a CD8⁺ T cell, or
(c) 12 minutes or faster in the case of a class II-restricted TCR transfected into a CD4⁺ T cell.

4. A method of treatment of a disease selected from cancer and infection comprising administering to a subject suffering such disease a plurality of TCR-transfected CD4⁺ and/or CD8⁺ T cells which are specifically activated by cells presenting a given pMHC characteristic of such disease, at least some of the transfected TCRs presented by each of said T cells having, in soluble form, a half-life for the interaction with the said pMHC within the range from 9.6 seconds to 12 minutes.

5. (canceled)


SEQUENCE LISTING (19 sequences)

SEQ ID NO: 1 - protein, 18 aa, Foot-and-mouth disease virus
LLNFDLLKLAGDVESNPG

SEQ ID NO: 2 - DNA, 822 bp, Homo sapiens
atggagaccctgctgggcctgctgatcctgtggctgcagctccagtgggtgtccagcaagcaggaggtgacccagatccctgccgccctgagcgtgcccgagggcgagaacctggtgctgaactgcagcttcaccgactccgccatctacaacctgcagtggttccggcaggaccccggcaagggcctgaccagcctgctgctgatccagagcagccagcgggagcagaccagcggacggctgaacgccagcctggacaagagcagcggccggagcaccctgtacatcgccgccagccagcccggcgacagcgccacctacctgtgcgctgtgcggcctaccagcggcggcagctacatccccaccttcggcagaggcaccagcctgatcgtgcacccctacatccagaaccccgaccccgccgtgtaccagctgcgggacagcaagagcagcgacaagtctgtgtgcctgttcaccgacttcgacagccagaccaatgtgagccagagcaaggacagcgacgtgtacatcaccgacaagaccgtgctggacatgcggagcatggacttcaagagcaacagcgccgtggcctggagcaacaagagcgacttcgcctgcgccaacgccttcaacaacagcattatccccgaggacaccttcttccccagccccgagagcagctgcgacgtgaaactggtggagaagagcttcgagaccgacaccaacctgaacttccagaacctgagcgtgatcggcttcagaatcctgctgctgaaggtggccggattcaacctgctgatgaccctgcggctgtggagcagc

SEQ ID NO: 3 - DNA, 933 bp, Homo sapiens
atgagcatcggcctgctgtgctgcgccgccctgagcctgctgtgggcaggacccgtgaacgccggagtgacccagacccccaagttccaggtgctgaaaaccggccagagcatgaccctgcagtgcgcccaggacatgaaccacgagtacatgagctggtatcggcaggaccccggcatgggcctgcggctgatccactactctgtgggagccggaatcaccgaccagggcgaggtgcccaacggctacaatgtgagccggagcaccaccgaggacttccccctgcggctgctgagcgctgcccccagccagaccagcgtgtacttctgcgccagcagctatgtgggcaacaccggcgagctgttcttcggcgagggctccaggctgaccgtgctggaggacctgaagaacgtgttcccccccgaggtggccgtgttcgagcccagcgaggccgagatcagccacacccagaaggccacactggtgtgtctggccaccggcttctaccccgaccacgtggagctgtcctggtgggtgaacggcaaggaggtgcacagcggcgtgtctaccgacccccagcccctgaaggagcagcccgccctgaacgacagccggtactgcctgtcctccagactgagagtgagcgccaccttctggcagaacccccggaaccacttccggtgccaggtgcagttctacggcctgagcgagaacgacgagtggacccaggaccgggccaagcccgtgacccagattgtgagcgccgaggcctggggcagggccgactgcggcttcaccagcgagagctaccagcagggcgtgctgagcgccaccatcctgtacgagatcctgctgggcaaggccaccctgtacgccgtgctggtgtctgccctggtgctgatggctatggtgaagcggaaggacagccggggc

SEQ ID NO: 4 - protein, 274 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 242 aa, Homo sapiens
MGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRAD

SEQ ID NO: 6 - DNA, 627 bp, Homo sapiens
atgcaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccacatcaggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 7 - DNA, 729 bp, Homo sapiens
atgggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 8 - protein, 208 aa, Homo sapiens
MQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 9 - protein, 242 aa, Homo sapiens; identical to SEQ ID NO: 5

SEQ ID NO: 10 - DNA, 822 bp, Homo sapiens
atgatgaagagcctgagggtgctgctggtgatcctgtggctgcagctgtcctgggtgtggagccagcagaaggaggtggagcagaatagcggccctctgagcgtgcccgagggcgccatcgccagcctgaactgtacctacagcgacagaggcagccagagcttcttctggtacaggcagtacagcggcaagagccccgagctgattatgttcatctacagcaacggcgacaaggaggacggcagattcaccgcccagctgaacaaggccagccagtacatcagcctgctgatccgggatagcaagctgtccgacagcgccacctacctgtgtgccgtgagaaccaatagcggctacgccctgaatttcggcaagggcaccagcctgctggtgaccccccacatccagaatcctgaccccgccgtgtaccagctgagagacagcaagagcagcgacaagagcgtgtgtctgttcaccgacttcgacagccagaccaacgtgtcccagagcaaggacagcgacgtgtacatcaccgacaagaccgtgctggacatgaggagcatggacttcaagagcaacagcgccgtggcctggagcaacaagagcgacttcgcctgtgccaacgccttcaacaacagcatcatccccgaggacacctttttccccagccctgagagcagctgtgacgtgaaactggtggagaagagcttcgagaccgacaccaacctgaacttccagaacctgagcgtgatcggcttcagaatcctgctgctgaaggtggccggattcaacctgctgatgaccctgagactgtggagcagc

SEQ ID NO: 11 - DNA, 930 bp, Homo sapiens
atgggacccggcctgctgtgctgggccctgctgtgcctgctgggagccggactggtggacgccggagtgacccagagccccacccacctgattaagaccaggggccagcaggtgaccctgagatgtagccctaagagcggccacgataccgtgtcctggtatcagcaggccctgggccagggaccccagttcatcttccagtactacgaggaggaggagaggcagagaggcaacttccccgacagattcagcggccaccagttccccaattacagcagcgagctgaacgtgaatgccctgctgctgggcgacagcgccctgtacctgtgtgccagcagcgacacagtgagctacgagcagtacttcggccctggcaccagactgaccgtgaccgaggacctgaagaacgtgttccctcctgaggtggccgtgttcgagcccagcgaggccgagatcagccacacccagaaggccaccctggtgtgtctggccaccggcttctaccccgaccacgtggagctgtcctggtgggtgaacggcaaggaggtgcacagcggcgtgtccaccgacccccagcccctgaaggagcagcccgccctgaacgatagcaggtactgcctgagcagcaggctgagagtgagcgccaccttctggcagaacccccggaaccacttcagatgccaggtgcagttctacggcctgagcgagaacgacgagtggacccaggatagagccaagcccgtgacccagatcgtgtccgccgaggcctggggcagagccgactgtggcttcaccagcgagagctaccagcagggcgtgctgtccgccaccatcctgtacgagatcctgctgggcaaggccacactgtacgccgtgctggtgtccgccctggtgctgatggctatggtgaagcggaaggacagcaggggc

SEQ ID NO: 12 - protein, 274 aa, Homo sapiens
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYISLLIRDSKLSDSATYLCAVRTNSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 310 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSDTVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - DNA, 630 bp, Homo sapiens
ccatcgatggcccagaaggaggtggagcagaattctggacccctcagtgttccagagggagccattgcctctctcaattgcacttacagtgaccgaggttcccagtccttcttctggtacagacaatattctgggaaaagccctgagttgataatgttcatatactccaatggtgacaaagaagatggaaggtttacagcacagctcaataaagccagccagtatatttccctgctcatcagagactccaagctcagtgattcagccacctacctctgtgcggtgcgcacaaattccgggtatgcactcaacttcggcaaaggcacctcgctgttggtcacaccccatatccagaaccctgaccctgccgtgtaccagctgagagactctaagtcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaa

SEQ ID NO: 15 - DNA, 742 bp, Homo sapiens
tctctcattaatggaggctggagtcacacaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcgacaccgtctcctacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaa

SEQ ID NO: 16 - protein, 207 aa, Homo sapiens
MAQKEVEQNSGPLSVPEGAIASLNCTYSDRGSQSFFWYRQYSGKSPELIMFIYSNGDKEDGRFTAQLNKASQYISLLIRDSKLSDSATYLCAVRTNSGYALNFGKGTSLLVTPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 17 - protein, 243 aa, Homo sapiens
MEAGVTQSPTHLIKTRGQQVTLRCSPKSGHDTVSWYQQALGQGPQFIFQYYEEEERQRGNFPDRFSGHQFPNYSSELNVNALLLGDSALYLCASSDTVSYEQYFGPGTRLTVTEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 18 - DNA, 3342 bp, Artificial Sequence
  pEX821 expression vector
gatctcgatcccgcgaaattaatacgactcactatagggagaccacaacggtttccctctagaaataattttgtttaactttaagaaggagatataatcgatgtctaactcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtgaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 19 - DNA, 3836 bp, Artificial Sequence
  pEX821 expression vector
gatctcgatcccgcgaaattaatacgactcactatagggagaccacaacggtttccctctagaaataattttgtttaactttaagaaggagatatacatatgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag